(19) United States
(12) Patent Application Publication
(10) Pub. No.: US 2018/0197424 A1
(43) Pub. Date: Jul. 12, 2018

(54) SYSTEM AND METHOD FOR COGNITIVE TRAINING

(71) Applicant: ACE APPLIED COGNITIVE ENGINEERING LTD, Hod Hasharon (IL)

(72) Inventors: Jacob GREENSHPAN, Tsur Moshe (IL); Dror LIVNAT, Ramat Hasharon (IL); Danny DANKNER, Kfar Shmaryahu (IL)

(73) Assignee: ACE APPLIED COGNITIVE ENGINEERING LTD, Hod Hasharon (IL)

(21) Appl. No.: 15/740,373

(22) PCT Filed: Jun. 28, 2016
(86) PCT No.: PCT/IL2016/050688

§ 371 Date: Dec. 28, 2017

Related U.S. Application Data
(60) Provisional application No. 62/187,339, filed on Jul. 1, 2015.

(51) Int. Cl. G09B 5/06; A61B 5/11; G09B 5/14; A61B 5/01

(52) U.S. Cl. CPC G09B 5/065; A61B 5/01; G09B 5/14; A61B 5/1118

(57) ABSTRACT

A personal wearable computing device and a method for cognitive training of a trainee is provided. The device comprising a CPU, a storage unit, input devices configured to take photos and video streams, to receive cognitive training input from a remote trainer and to receive commands and responses from said trainee and output devices configured to present tasks and instructions to said trainee and to send visual and auditory and the trainee's performance representation of the vicinity of said trainee to said trainer. The method comprising providing cognitive training data and instructions to a personal wearable computing device, receiving response and performance data from said at least one trainee and storing and processing said performance data and providing updated cognitive training instructions, in response to said received response and performance data.

### BACKGROUND OF THE INVENTION

Cognitive training aided by computerized systems is known, and particularly when used to enhance performance in a specific assignment (such as flight, sport or medical activity). However such computerized cognitive training system typically presents tasks to the trainee in an environment that is different and/or remote from the addressed assignment; hence the trainee reacts only to the task posed to him by the training system, with no reference to real life events taking place in the immediate vicinity of the trainee.

There is a need for computerized cognitive training system and methods for training a trainee whilst in his/her target acting environment or within his/her simulated environment, for example where or when such cognitive training target environment is not desirable. For example, cognitive training a commercial pilot while flying a commercial Jet is not an option due to the highly risk for lives and equipment. Alternatively such commercial pilot may be trained in a high fidelity simulator. In another example, an athlete may be trained while he/she is engaged in his/her sport typical activity, in the sport's typical field or yard by presenting to the trainee tasks and events requiring his/her reaction with respect to the computer produced task considering and relating to real-time events taking place in his immediate vicinity. Such training can take place using wearable technology.

### SUMMARY OF THE INVENTION

A personal wearable computing device for cognitive training of a trainee is provided, the device comprising a CPU, a storage unit, input devices configured to take photos and video streams, to receive cognitive training input from a remote trainer and to receive commands and responses from said trainee and output devices configured to present tasks and instructions to said trainee and to send visual, auditory and the trainee's performance representation of the vicinity of the trainee to the trainer.

According to embodiments of the invention the instructions from the trainer are sent in response to the representation of the vicinity and the trainee's performance.

According to additional embodiment the instructions from the trainer are stored in a remote storage and are sent from the storage to the trainee in response to the trainee's inputs during the cognitive training.

A system for cognitive training is disclosed comprising at least one personal wearable computing device comprising a CPU, a storage unit, input devices configured to take photos and video streams, to receive cognitive training input from a remote trainer and to receive commands and responses from said trainee and output devices configured to present tasks and instructions to said trainee and to send visual and auditory and the trainee's performance representation of the vicinity of said trainee to said trainer. The system further comprising at least one peripheral device for reflecting events taking place in the cognitive training field and at least one peripheral device for providing instructions and data to at least one cognitive trainee in the cognitive training field.

According to embodiments of the present invention the system further comprising central server configured to store, process and provide data and training instructions to the at least one personal wearable computing device.

According to embodiments of the invention the central server is further configured to receive representation of the vicinity of the at least one trainee and representation of the performance of said at least one trainee, and to process and provide the at least one trainee data and instructions in response to said trainee's performance and input data.

A method for cognitive training of at least one trainee is disclosed comprising: providing cognitive training data and instructions to a personal wearable computing device, receiving response and performance data from the at least one trainee and storing and processing the performance data and providing updated cognitive training instructions, in response to said received response and performance data.

According to embodiments of the invention the instructions that are provided to personal wearable computing device are in response to performance and response received from at least one other personal wearable computing device.

According to embodiments of the invention method further comprising instructing the at least one trainee to respond to a task by making reference to one other trainee of the at least one trainee.

According to embodiments of the invention the instructions provided to the personal wearable computing device are processed in a remote computing service prior to being provided.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a high level block diagram of a computing device according to embodiments of the present invention;

FIG. 2 is a schematic illustration of a system for performing cognitive training to trainee in his/her natural field of activity, according to embodiments of the present invention;

FIG. 3A is a schematic block diagram depicting a training field according to embodiments of the present invention;

FIG. 3B is a schematic block diagram depicting another training field according to embodiments of the present invention; and

FIG. 4 is a schematic flowchart depicting a method for real time cognitive training according to some embodiments of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

Cognitive training system and methods according to embodiments of the present invention may be configured to provide cognitive stimuli and to receive record and assess responses of a trainee to the stimuli. The stimuli and responses may be provided and recorded whilst the trainee is acting within his/her natural activity environment, exposed to events taking naturally place in such environment and be operated to react to training tasks making reference to objects acting or actions taking place in his/her immediate environment. All this activity may be done in a manner that embeds the training tasks into the trainee's natural operating environment. For example, a sportsman trainee training with his team members on a game field may be asked by a portable device operative according to embodiments of the present invention and carried by the trainee, to mark the relative location of a certain team member with respect to the trainee by indicating the relative direction via input means of the portable device. Other tasks may be presented to the trainee, where they impose substantially same cognitive processing resources load of the above real life task, or at least impose equivalent cognitive load. For example the portable device may be a wearable computation and communication device allowing receiving and sending data over wireless communication channels, storing and processing programs and data items and presenting data and instructions to a user and receiving from the user input indications. The wearable device may further comprise location and orientation indicators.

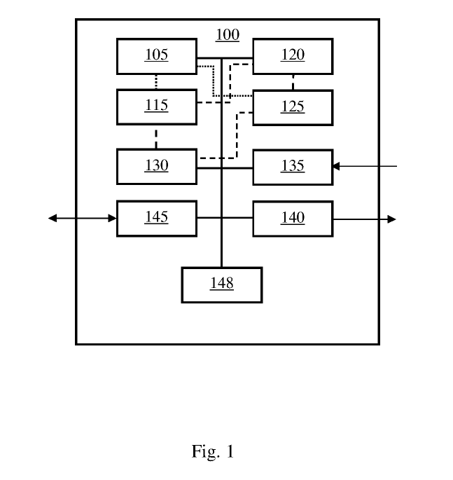

Reference is made to FIG. 1, showing a high level block diagram of an exemplary computing device 100 according to embodiments of the present invention. Computing device 100 may include a controller 105 that may be, for example, a central processing unit processor (CPU), a chip or any suitable computing or computational device, an operating system 115, a memory 120, an executable code 125, a storage 130, input devices 135, output devices 140, communication unit 145 and status sensors unit 148. Controller 105 may be configured to carry out methods described herein, and/or to execute or act as the various modules, units, etc. According to some embodiments certain portions of the computing may be done out of device 100. For example, some computation may be done remotely, for example by a cloud computing service (not shown). More than one computing device 100 may be included in a system according to embodiments of the invention, and one or more computing devices 100 may act as the various components of a system. For example, each of the portable devices described herein may be, or may include components of computing device 100. For example, by executing executable code 125 stored in memory 120, controller 105 may be configured to carry out a method of providing cognitive training of a trainee.

Operating system 115 may be or may include any code segment (e.g., one similar to executable code 125 described herein) designed and/or configured to perform tasks involving coordination, scheduling, arbitration, supervising, controlling or otherwise managing operation of computing device 100, for example, scheduling execution of software programs or enabling software programs or other modules or units to communicate. Operating system 115 may be a commercial operating system.

Memory 120 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 120 may be or may include a plurality of, possibly different memory units. Memory 120 may be a computer or processor non-transitory readable medium, or a computer non-transitory storage medium, e.g., a RAM.

Executable code 125 may be any executable code, e.g., an application, a program, a process, task or script. Executable code 125 may be executed by controller 105 possibly under control of operating system 115. For example, executable code 125 may be an application that manages a portable device as further described herein. Although, for the sake of clarity, a single item of executable code 125 is shown in FIG. 1, a system according to embodiments of the invention may include a plurality of executable code segments similar to executable code 125 that may be loaded into memory 120 and cause controller 105 to carry out methods described herein. For example, units or modules described herein may be, or may include, controller 105 and executable code 125.

Storage 130 may be or may include, for example, a hard disk drive, a solid state drive (SSD), solid state (SD) card, a Blu-ray disk (BD), a universal serial bus (USB) device or other suitable removable and/or fixed storage unit, configured to store non-transient data executable by controller 105. Content may be stored in storage 130 and may be loaded from storage 130 into memory 120 where it may be processed by controller 105. In some embodiments, some of the components shown in FIG. 1 may be omitted. For example, memory 120 may be a non-volatile memory having the storage capacity of storage 130. Accordingly, although shown as a separate component, storage 130 may be embedded or included in memory 120.

Input devices 135 may be or may include data input means such as touch screen, keypad, keyboard and the like, capable of inputting data and control commands, or any suitable input device. It will be recognized that any suitable number of input devices may be operatively connected to computing device 100 as shown by block 135.

Output devices 140 may include one or more displays or monitors, speakers, vibrating means, visual, auditory and tactile means and/or any other suitable output devices. It will be recognized that any suitable number of output devices may be operatively connected to computing device 100 as shown by block 140. Any applicable input/output (I/O) devices may be connected to computing device 100 as shown by blocks 135 and 140.

Communication unit 145 may be any unit allowing bidirectional communication of device 100 with other devices, for transmitting and receiving data, control commands and the like. The communication handled by communication unit 145 may be performed over wired system, may be wireless or may be carried over mixed communication infrastructure. Typically the communication may be wireless, using any known means and protocols such as WiFi, BlueTooth, infrared (IR) communication and the like.

Status sensors unit 148 may comprise one or more status indicators, sensors and measurement devices capable of sensing and indicating movements, location changes, orientation status, temperature (whether ambient temperature, in-device temperature, temperature of the user of device 100 or any combination thereof), images (such as a camera), heart rate, direction of look sensor such as smart glass, and the like.

Computing device 100 may be, according to embodiments of the invention, a wearable device having physical size and weight which are negligible with respect to the expected negative effect on the trainee's performance during his/her cognitive training. Accordingly, such embodiment of computing device 100 may be worn on the trainee's wrist, on the trainee's neck, on the trainee's cloth, and the like. Such embodiment of computing device 100 may be configured to provide audible and/or visible data and instructions to the trainee and may further be configured to receive response, data and any other type of input from the trainee. For example, computing device 100 may be worn on the trainee's wrist, may be configured to present data and instructions on the device's screen and may be configured to receive input from the trainee through the touch screen of device 100. According to some embodiments of the present invention device 100 may be embodied as part of a smartphone, using the smartphone's input, output and position and orientation means, its computing and storage means and its communication means.

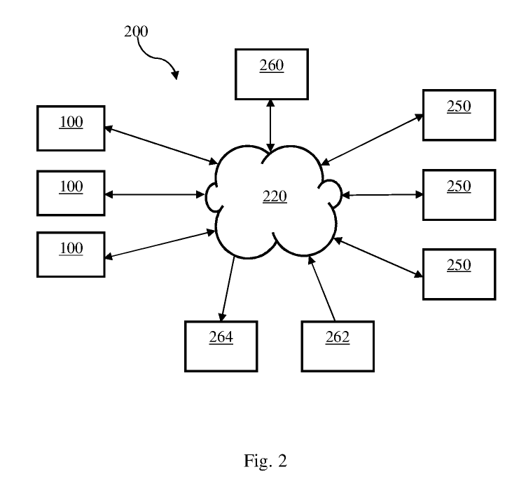

Device 100 may be in operative communication with other devices for receiving data, control commands and the like, and transmit data and/or control commands to other devices. Reference is made now to FIG. 2, which is a schematic illustration of system 200 for performing cognitive training to trainee in his/her natural field of activity, according to embodiments of the present invention. System 200 may comprise one or more computing devices such as computing device 100 in operative communication with one or more training control units 250, via communication network 220. System 200 may further comprise central server 260 for storing, processing and providing data derived from collections of training data received from a plurality of trainees. System 200 may further comprise one or more input units 262 and one or more output units 264. Input units 262 and output units 264 may be in operative communication with one or more computing devices 100, with one or more control units devices 250 and with central server 260, for example via network 220.

Input unit 262 may be or may include cameras to provide images and/or video streams reflecting events taking place at the vicinity of a trainee. Such reflected events may be operations and/or movements performed by the trainee, and/or movements or operations performed by other persons in the vicinity of the trainee (for example other players participating in the same game as the trainee). The reflected events may further include movements of objects related to the training session, such as the movement and momentary location of a ball in the football game. For example, a ball may be provided with any appropriate transponder allowing an external device located for example near the borders of the field to receive location indications of the ball at any time. It would be apparent that input devices (i.e., units) may comprise other means for reflecting events on the training site, as is known in the art.

Output unit 264 may be or may comprise audible means such as loudspeakers, publically or personally visible means such as screens, illumination/light-based indicators, and the like. Output unit 264 may be configured to provide audible and/or visible data and commands for example to a trainee. According to some embodiments output unit may be configured to provide data such as audible instructions, visible text and/or visible indications receivable anywhere in the field where the trainee is active during the training. For example, for a trainee who is a football player training to improve his/her reaction time, during his regular football training session, certain cognitive training tasks may be communicated to the trainee while he/she is on the football field via loudspeaker or large screens Similarly, the performance of the trainee may be communicated to system 200 using one or more cameras photographing (or video recording) the trainee and the football field which are within the cameras field of view. Other input sensors may be short range wireless transceivers located around the football field, or other wireless communicating means that are not located on the trainee but are capable of providing performance indications of the trainee to system 200.

Training control units 250 may be any suitable computing unit such as desktop computer, laptop computer, note book computer, smartphone and the like, capable of storing and executing software programs according to embodiments of the present invention. Training control units 250 may further be configured to communicate with one or more computing devices 100 for providing training instructions and tasks, for receiving indications of the performance of the training tasks and for providing further training instructions responsive to the information representing the trainee's performance.

Central server 260 may be any suitable computing unit capable of receiving data from computing devices 100 and from training control units 250. Central server 260 may further be configured to store the received data, to sort the data, to process the data for calculating big data bases representing average performance, performance trends, performance records and the like. The big data based may be useful as a basis for analyzing personal or team performance and for forming new/fresh/updated training tasks.

Known methods and training schedules typically present stimuli and tasks to a trainee according to a scheme that is configured to fit conditions, ambient and circumstances that tend to simulate circumstances and conditions similar to, or as-close-as-possible to those pertaining to the field of regular activity of the trainee. While there are known advantages to training in simulated conditions, there are also certain disadvantages in this method of training, such as the inherent differences between the real world and the simulated world. T in a simulated environment may cause adoption of reaction schemes which perfectly fit the simulated conditions but deviates from the required reaction matching the respective real world. In some cases, the simulated environment only rarely presents the actual diversity of events, inter-timing between the events and the load imposed on the trainee due to these events, to name only few.

Systems and methods according to embodiments of the present invention allow for cognitive training within the real world of activity of the trainee and within the real world of events of that trainee, thus overcoming the disadvantages of known training methods and systems.

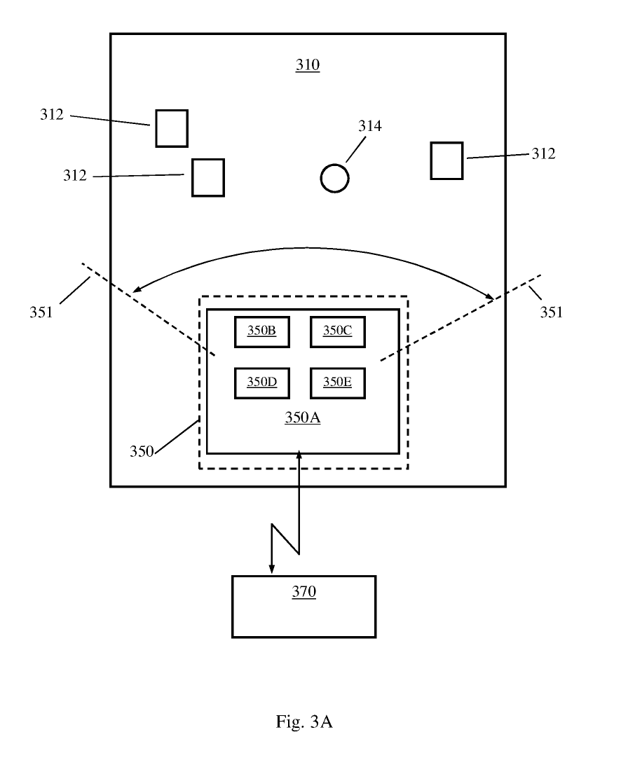

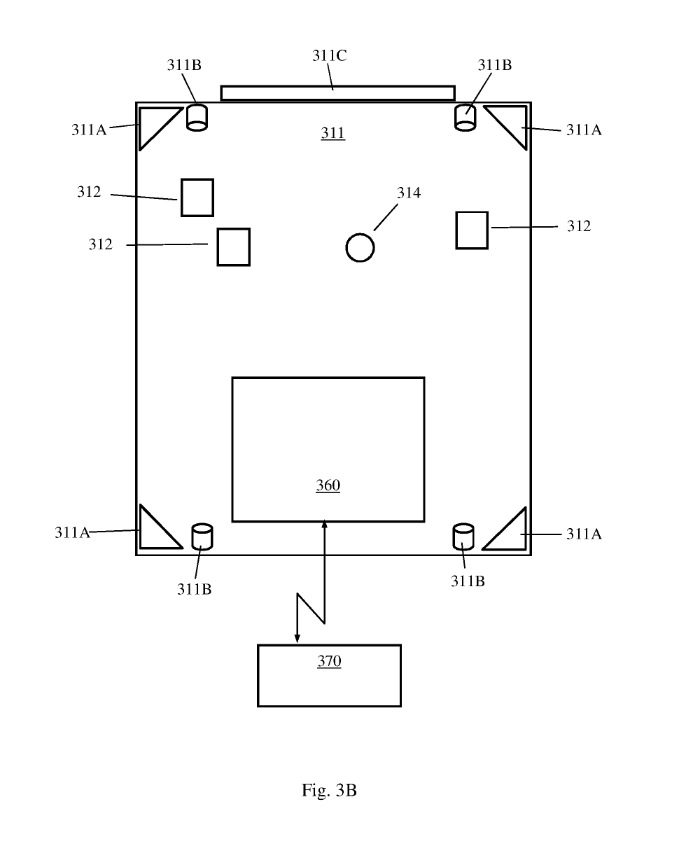

In some embodiments, in order to provide a cognitive trainee with real time training instructions the trainee may act in a training field provided with appropriate means allowing to reflect events taking place in the field to a cognitive trainer and means for providing instructions to the trainee, for example in response to such events. Reference is made now to FIG. 3A which is a schematic block diagram depicting training field 310 according to embodiments of the present invention. Training field 310 may be a real activity field of the respective trainee 350, where additional players 312 may be active and other objects 314 may be involved. For example, for a trainee 350 who is a football player, field 310 may be a real football field, players 312 may be other players in trainee's 350 team and/or players of the opposing team and object 314 may be the football. Trainee 350 may be provided computing unit 350A provided with field sensing means such as one or more cameras 350B and optionally one or more microphones 350C capable of sensing and reflecting visual and audible events taking place at least within the events sensing range depicted by dashed lines 351. Computing unit 350A may be carried on or by trainee 350. Computing unit 350 may further comprise visual means 350D and/or audible means 350E capable of providing dynamic instructions to trainee 350 while participating in a real game, as described in details with reference to FIG. 1. Instructions sent to trainee 350 by trainer 370 and field data sensed by sensing means 350B and/or 350C may be exchanged between trainee 350 and trainer 370 over a wireless network as is known in the art and described in details with reference to FIGS. 1 and 2. Trainer 370 may follow and assess the reactions of trainee 350 to events in the filed in combination with the way trainee 350 carries out training instructions and tasks sent to him, and may dynamically adjust and modify existing training instructions and tasks or provide new ones, in order to optimize the training session in terms of training required achievements. See detailed schemes of dynamic and real time cognitive training according to embodiments of the present invention herein below.

Reference is made now to FIG. 3B, which is a schematic block diagram depicting training field 311 according to embodiments of the present invention. Training field 311 may be configured to provide reflection of events in the training field to a trainer 3770 and to provide training instructions and tasks to a trainee 360 in case the trainee does not use a wearable computing unit, such as unit 350A of FIG. 3A. According to embodiments of the present invention training field 311 may be provided with one or more video sensing means such as video cameras 311A capable of sensing visual events in field 311 and sending same to trainer 370. For example, video cameras 311A may detect the movements of trainee 360, one or more players 312 and ball 314. Training field 311 may further include one or more audible means such as loudspeakers 311B and at least one large screen 311C, capable of sensing visual events in field 311 and sending same to trainer 370 and further capable of providing visual and audible instructions from trainer 370 to trainee 360 via screen 311C and loudspeakers 311B. It would be apparent to those skilled in the art that any suitable combination of sensing units or devices and instructions giving units or devices with wearable means may be used for providing dynamic and real time reflection of the events in the field and for providing real time instructions to the trainee may be used.

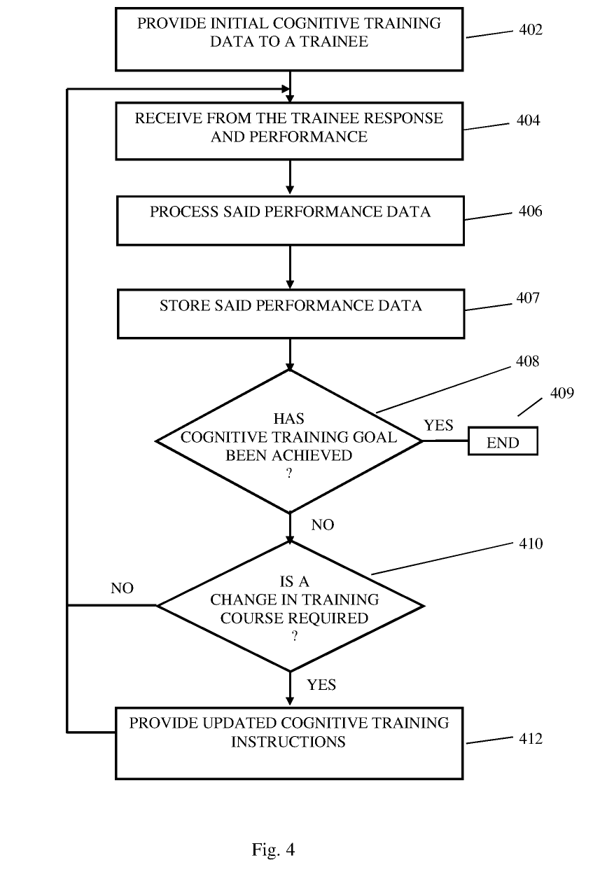

Reference is made now to FIG. 4, which is a schematic flowchart of a method of real time cognitive training of at least one trainee according to some embodiments of the present invention. In block 402 initial cognitive training data and instructions (e.g., tasks) may be provided to the trainee via a personal computing device (e.g., wearable device 100). In block 404 response and performance data from said at least one trainee may be received. The performance data may include real time indications from the trainee reflecting events taking place in the field of training and the trainee's performance may be provided to the trainer, for example, via system such as system 200. The trainer may process the performance by, for example, evaluating, assessing and weighing the trainee's performance in conjunctions with the real time situation and events in the training field in block 406. In block 407 the processed data may be stored, for example, in storage unit 130.

In decision point 408 the trainer may decide whether the cognitive training goals have been achieved. In case the cognitive training goals have been achieved the cognitive training session may terminate (block 409). In case the cognitive training goals have not been achieved at decision point 410 the trainer may decide whether a change in the training instructions/tasks should be made. In case no change is required the trainee proceeds his/her training according the instructions/tasks provided previously (block 404). In case a change is required updated set of cognitive training instructions (e.g., tasks) may be provided to the trainee (block 412) and the training session continues with new/updated set of cognitive training instructions/tasks.

The cognitive training instructions/tasks formed by the trainer may be configured to match various required achievements and according to various training methods, as described in details herein below.

Cognitive Training Modules for a Trainer/Coach

It would be apparent to those skilled in the art that when a cognitive trainee is a member in a team, such as in sports (e.g. football) in the cognitive training modules presented below as examples the trainer may be one of the training staff of the team. One example of a cognitive training module may be a module providing control of the training in the training field. For example the trainer may set a cognitive training program for a specific trainee, associated with a specific training field for a specific training period of time going forward. Such program may take advantage of indications of events taking place in the training field and provide dynamically changing cognitive program in response to such indications.

Another training module may be formed for cognitive training outside of the training field. For example cognitive tasks may be given to the trainee while he is commuting to the training field or commuting home, for example on a subway train coach, via his smartphone. Training during commuting may take advantage of data related to cognitive training of the commuting user as collected and optionally analyzed during the recent training or trainings according to embodiments of the present invention. Another cognitive training module may allow a cognitive trainer to debrief the trainee's performance after a training session by “playing back” record of the training session and analyzing the trainee's performance with the trainee, with the trainee's team members and/or the trainee's training staff.

In an additional or alternative embodiment debriefing of the training session may be done with the assistance of video streams taken by cameras covering the training field, such as cameras 311A of FIG. 3B. In additional or alternative embodiments such data may also be collected by computing devices carried or worn by colleagues (e.g., other players) of the trainee.

In an additional or alternative embodiment the trainer may inject changes in the training session of the trainee at selected time and impose a selected change in the training session. For example the trainer may add, at real time, task to be performed immediately by the trainee, thus imposing additional burden on the trainee's attention resources. The system and methods according to embodiments of the present invention may allow the trainer to select the exact time this added task is presented to the trainee, as well as the nature of that task, thus enabling the trainer to confront the trainee with as accurate as possible tasks in order to fine tune the cognitive training. According to yet additional embodiment the trainer may ‘inject’ at a selected time a set of missions to two or more of the team members thus forming an even more complicated controlled situation to which the team members need to react, thus providing a better and more accurate cognitive training tasks.

Cognitive Training Modules for a Trainee

A system and method according to embodiments of the present invention may be used for enhancing and improving the cognitive training of a trainee by relying on real time indication of the performance of the trainee and the situation of the trainee within a real field of activity and further by enabling providing of cognitive training instructions and missions responding in real time to these indications. The following are examples of cognitive training schemes performed using a system and methods according to embodiments of the present invention.

In one cognitive training scheme a trainee may be asked to perform, while being busy in his real world activity a specific action in response to the location of certain team members of his team in the field. The response to this instruction may be entered to the system by any suitable input device as detailed above. In another embodiment the trainee may be instructed to turn his sight to the direction of the fastest player on the training field when a ball is thrown. This may assist in training the trainee to locate the fastest player in transient situations on the field. In training activities involving potential risks when cognitive training may impose high attention loads, such as where the trainee is a pilot, the training program may be carried out in a high fidelity simulator, thus practicing the pilot to deal with high attention loads, in order to maintain training conditions as-close-as-possible to real life while reducing the risk of undesired results due to reduction of the trainee's attention to his/her routine activities.

Another type of cognitive training may be configured for indirectly loading the trainee by instructing him to perform tasks which are not directly related to his/her on-going mission. For example, instructing the trainee to perform a certain movement when predefined visual indication is presented to him/her on the wearable device, thus loading his attention with a mission that is not directly related to his on-going action. In yet another example the trainee may be requested to point at a location of a team member of the opposing team that has crossed the trainee's field of view two seconds ago and presently is not in his FOV.

In yet another example, indicators capable of continuously indicating the location and movement of the trainee may be used. For example the trainee may be instructed to change direction of movement, or direction of sight, when his location was indicated to be at a predefined location in the training field. In another embodiment the trainee may be requested to indicate, via his wearable computing device, the relative location of a certain player in the training field. In yet another embodiment the trainee may be requested to enter the number of opponent players in the field the distance of which from the trainee is no more than a certain distance.

System and method operative according to embodiments of the present invention may provide training tasks based on tracing of the trainee's movement in the training field. For example, the trainee may be asked to follow a time plan for running a certain distance while changing the time-to-target occasionally, thus forcing the trainee to re-calculate the proper running speed while running. In yet another embodiment, the trainee may be instructed to provide, during his training session, indication of player in the field who is the fastest to reach another certain player. In yet another example, a trainee who practicing driving a car in a car driving simulator may be requested to evaluate the number of cars that could be involved in a car accident, should his own car was not stopped and thus causing the car accident. It should be noticed that training a trainee in a high fidelity simulator may still involve differences between the simulated conditions and terms and the real life conditions and terms. Such differences should be taken in considerations when planning the training sessions.

In other embodiments of the present invention training may be based on the movement of the trainee in the training field. For example, the trainee may be instructed to indicate, at a certain moment of activity in the training field, who is the team member on the field who is the fastest to run towards a certain player in the opponent team. This may be helpful in training the trainee to snap movement patterns of objects in his/her field of training.

According to some embodiments the trainee may be instructed to take an action when change in the flow of the sport game took place. For example, the trainee may be instructed to take a certain action when his team lost the ball. In such a situation the system may sense by identifying the majority of the players on the field that may be suddenly moving towards the side of field associated with the team of the trainee.

In yet other embodiments the trainee may be instructed at a certain time to stop his/her activity associated with the events on the field and perform mathematical calculations presented to him/her. This kind of training forces the trainee to rapidly disconnected his attention from events in the field and pay attention to data presented to him via his computing unit or via peripheral units, thus improving the trainee's situational awareness and task switching capabilities.

According to embodiments of the present invention the trainee may be instructed, at a certain time during his/her training activity, to indicate via his/her input device, the relative direction of movement of a certain player on the field thus training the trainee to pay better attention to movements around him.

According to other embodiment the trainee may be instructed to immediately make predefined certain movement in a predefined certain direction when a certain predefined input is presented to him/her. This may assist in improving the trainee's capability of holding more than one action plan concurrently.

The examples given above and any other embodiment pertaining to the present invention may rely on measurement and assessment of the trainee's movements and reactions as measured and provided via movement and location indicators carried by the trainee, for example using wearable(s) and/or as measured and derived via peripheral input means such as one or more cameras located at the perimeter of the activity field of the trainee. The trainee may provide input as part of his/her tasks and missions using input devices on his wearable device, on computing means carried by him or using video interpreting software deriving the trainee's gestures as caught in the video frames.

In certain embodiments a wearable computing device formed as a wrist watch may be used as an input means where relative directions in the field may be presented by a relative movement of a finger over the screen of the wearable device, the “ENTER” input may be associated with a long pressing on a fixed location on the screen, as is known in the art. According to some embodiments a speech to data converter software may be used for receiving the trainee's input by interpreting his/her voice inputs.

According to some embodiments of the present invention actions taken by the trainee and physiological parameters of the trainee may be collected and transmitted to the system in order to be used as enriched data base of the trainee's performance. The physiological parameters, such as blood pressure, heart rate, body temperature and the like may be measured using wearable instruments as is known in the art. Such input parameters may be used to set points of intervention of the trainer, or to set points of changing of the cognitive tasks responsive of the trainee's level of physical effort, physical fatigue and the like. For example, when a raise in the level of fatigue is identified the cognitive training scheme may be changed by adding awareness load aiming to improve the capability of the trainee to take complicated decisions under high effort and high level of fatigue.

The system and methods described above may also provide for personalization of the trainee's training profile, based accumulation of the trainee's performance along time. Since the training is performed very close to real-life conditions, and mostly within real-life conditions, the accumulated trainee's performance results serve as best grounds for fine tuning of the trainee's future training sessions, aiming to achieve better results.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A personal computing device for cognitive training of a trainee comprising:
a central processing unit (CPU);
a storage unit;
input devices configured to take photos and video streams, to receive cognitive training input from a remote trainer and to receive commands and responses from said trainee; and
output devices configured to present tasks and instructions to said trainee and to send visual and auditory and the trainee's performance representation of the vicinity of said trainee to said trainer.

2. The personal computing device of claim 1, wherein the device is a wearable device.

3. The personal computing device of claim 1 or claim 2, wherein said instructions from said trainer are sent in response to said representation of the vicinity and the trainee's performance.

4. The personal computing device according to any one of claims 1-3, wherein said instructions from said trainer are stored in a remote storage and are sent from said storage to said trainee in response to the trainee's inputs during the cognitive training.

5. The personal computing device according to any one of claims 1-4, wherein the central processing unit is configured to:
receive from the input devices information related to at least one of: a location of the trainee in a training field, locations of one or more players in the play field and a location of an object in the training field; and
send the information to the trainer via a communication unit.

6. A system for cognitive training comprising:
at least one personal computing device comprising:
a CPU; and
a storage unit;
at least one peripheral device for reflecting events taking place in a cognitive training field; and
at least one peripheral device for providing instructions and data to at least one cognitive trainee in the cognitive training field.

7. The system of claim 6, wherein the personal computing device is a wearable device comprising:
input devices configured to take photos and video streams, to receive cognitive training input from a remote trainer and to receive commands and responses from said trainee; and
output devices configured to present tasks and instructions to said trainee and to send visual and auditory and the trainee's performance representation of the vicinity of said trainee to said trainer.

8. The system of claim 6 or claim 7, further comprising a central server configured to store, process and provide data and training instructions to said at least one personal wearable computing device.

9. The system of claim 8 wherein said central server is further configured to receive representation of the vicinity of said at least one trainee and representation of the performance of said at least one trainee, and to process and provide said at least one trainee data and instructions in response to said trainee's performance and input data.

10. A method for cognitive training at least one trainee comprising:
providing cognitive training data and instructions to a personal wearable computing device;
receiving response and performance data from said at least one trainee and storing and processing said performance data; and
providing updated cognitive training instructions, in response to said received response and performance data.

11. The method of claim 10, wherein said instructions provided to said personal wearable computing device are in response to performance and response received from at least one other personal wearable computing device.

12. The method of claim 10 or claim 11, further comprising instructing said at least one trainee to respond to a task by making reference to one other of said at least one trainee.

13. The method according to any one of claims 10-12 wherein said instructions provided to said personal wearable computing device are processed in a remote computing service prior to being provided.

14. The method according to any one of claims 10-13, wherein receiving response and performance data from said at least one trainee comprises information related to at least one of: a location of the trainee in a training field, locations of one or more players in the play field and a location of an object in the training field.
